(19) United States
(12) Patent Application Publication
(10) Pub. No.: US 2013/0059062 A1
(43) Pub. Date: Mar. 7, 2013

(54) Device For The Manufacture Of A Dosage Form With A Hole And Method Of Manufacture

(71) Applicants: Ramakant Kashinath Gundu, Ahmednagar (IN); Rahul Sudhakar Dabre, Nagpur (IN); Girish Kumar Jain, Delhi (IN)

(72) Inventors: Ramakant Kashinath Gundu, Ahmednagar (IN); Rahul Sudhakar Dabre, Nagpur (IN); Girish Kumar Jain, Delhi (IN)

(21) Appl. No.: 13/582,452

(22) PCT Filed: Mar. 7, 2011
(86) PCT No.: PCT/IB2011/050939

§ 371 Date: Nov. 12, 2012

(30) Foreign Application Priority Data
Mar. 11, 2010 (IN) 649/MUM/2010

(51) Int. Cl. B28B 7/18; A61K 9/28

(52) U.S. Cl. 427/2.14; 425/352

(57) ABSTRACT

This invention is related to a device for the manufacture of a dosage form with a hole and method of manufacture. The dosage form may be a modified release dosage form comprising a core coated with a polymeric coat comprising one or more rate controlling polymers, said dosage form having a hole extending through the dosage form resulting in an inner radial surface and an outer radial surface, said core comprising at least one therapeutically active ingredient, characterized in that the inner radial surface is partially coated with said polymeric coat.

### FIELD OF THE INVENTION

This invention is related to a device for the manufacture of a dosage form with a hole and method of manufacture. The dosage form may be a modified release dosage form comprising a core coated with a polymeric coat comprising one or more rate controlling polymers, said dosage form having a hole extending through the dosage form resulting in an inner radial surface and an outer radial surface, said core comprising at least one therapeutically active ingredient, characterized in that the inner radial surface is partially coated with said polymeric coat.

### BACKGROUND OF THE INVENTION

The present invention provides a device and a method of manufacture of a dosage form having a hole wherein the dosage form polymeric coating would be partial on the hole portion of the dosage form. Known methods of manufacture of tablet with a hole that has a partial coating on its hole portion require costly and precision equipments such as mechanical drilling, use of ultrasonic technology and laser drilling.

U.S. Pat. No. 4,153,399 relates to multiple punch tool set for powder compacting press.

U.S. Pat. No. 5,897,887 relates to machine for the formation dosage forms of cosmetic product.

US application No. 20030029334 relates to punch and die using cobalt alloy for preparing tablets.

U.S. Pat. No. 5,376,771 discloses process for producing pharmaceutical drug delivery dosage form having plurality of apertures in its outside surface, the apertures providing an egress for a drag when the drug delivery device is placed in a biological environment of use, the process uses digital laser marking system.

The prior art modified release dosage forms with a central perforation or hole for releasing therapeutically active ingredients generally release a disproportionate amount of therapeutically active ingredients quickly after ingestion by a patient and fail to provide consistent release profile. The technology used in developing most of such dosage forms involves compression of tablet core followed by creating a perforation or hole in the coated or uncoated core by drilling or other means. These dosage forms provide brisk release of active ingredient through the perforation or hole instead of a consistent zero order release kinetics. Such systems are prone to variation in release kinetics and may result in a quick spike in the level of medication in the patient's bloodstream. However, there are many instances where a spike in the medication level in a patient is undesirable, as where pharmaceuticals are used to treat a chronic condition. Further, extended release of therapeutically active agents is highly desirable for agents that have characteristically short half-lives.

The cost involved in producing the prior art dosage forms often use expensive techniques and precision equipments such as laser drilling, mechanical drilling, ultrasonic technology and the like. Therefore there exists a need for a dosage form that will provide desired consistent release kinetics with a good bioavailability of therapeutically active pharmaceutical ingredients of various classes along with increased duration of action and decreased frequency of dosing.

The need for modified release dosage forms is particularly of importance in case of drugs such as lamotrigine, which is rapidly and completely absorbed after oral administration with negligible first pass metabolism. The immediate release formulations of lamotrigine often produce brisk release of lamotrigine in the stomach resulting in cyclical plasma concentration and pharmacokinetic profile with peaks occurring after administration followed by troughs occurring before the next administration of drug.

### SUMMARY OF THE INVENTION

The present inventors while working for suitable devices for the manufacture of dosage form having a hole has felt there is a need for (i) easily adaptable modification on few essential components of the existing tablet machine (ii) Minimum cost of modification (iii) easily replaceable components, (v) manufactured dosage form having a hole would have partial polymeric coating on its hole portion.

In one of the general aspect of the invention the present invention provides a device for the manufacture of a dosage form having a hole wherein the dosage form inner radial surface polymeric coating would be partial on the hole portion of the dosage form.

In another general aspect of the invention there is provided a method of manufacture of modified release dosage form comprising a core coated with a polymeric coat comprising one or more rate controlling polymers, said dosage form having a hole extending through the dosage form resulting in an inner radial surface and an outer radial surface, said core comprising at least one therapeutically active ingredient, characterized in that the inner radial surface is partially coated with said polymeric coat.

The details of one or more embodiments of the inventions are set forth in the description below. Other features, objects and advantages of the invention will be apparent from the description and claims.

### DETAILED DESCRIPTION OF THE INVENTION

The invention is described in detail with references to the following figures.

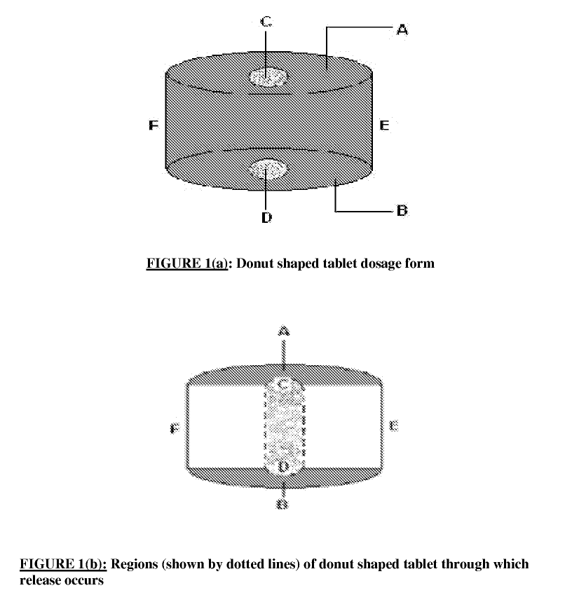

FIG. 1(a) is a general view of a dosage form designed as donut shaped tablet for oral administration of therapeutically active pharmaceutical ingredient;

FIG. 1(b) shows the partially coated region extending from C to D of inner surface of donut shaped tablet;

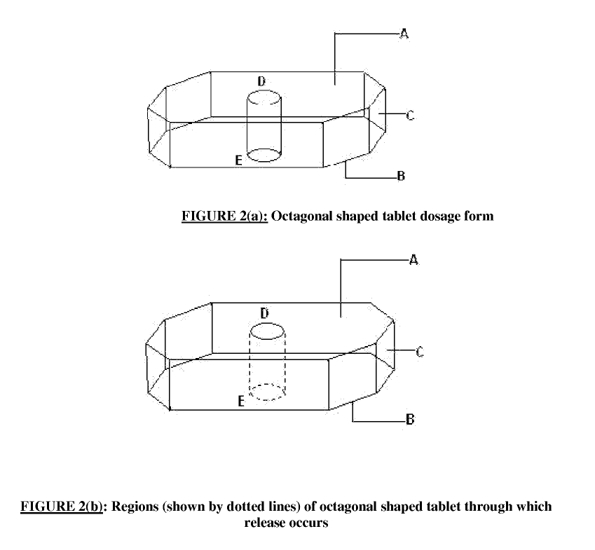

FIG. 2(a) is a general view of a dosage form designed as octagonal shaped tablet for oral administration of therapeutically active pharmaceutical ingredient;

FIG. 2(b) shows the regions (shown by dotted lines) of inner surface of octagonal shaped tablet;

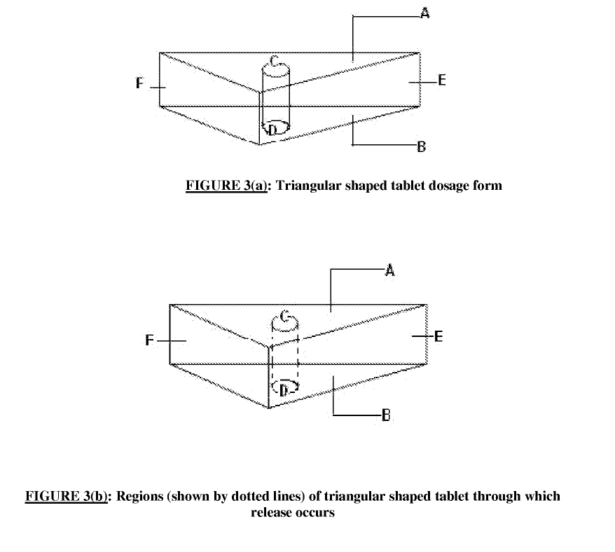

FIG. 3(a) is a general view of a dosage form designed as triangular shaped tablet for oral administration of therapeutically active pharmaceutical ingredient;

FIG. 3(b) shows the regions (shown by dotted lines) of inner surface of triangular shaped tablet through which controlled release of therapeutically active pharmaceutical ingredient occurs;

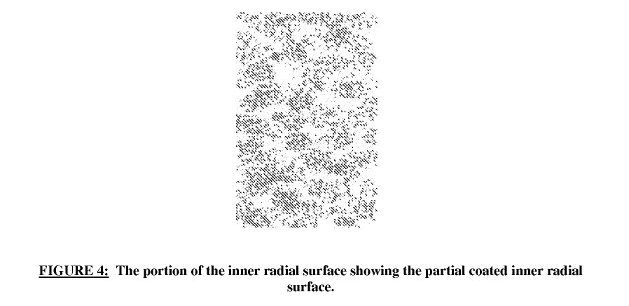

FIG. 4 shows the enlarged portion of the inner radial surface showing the partially coated inner radial surface;

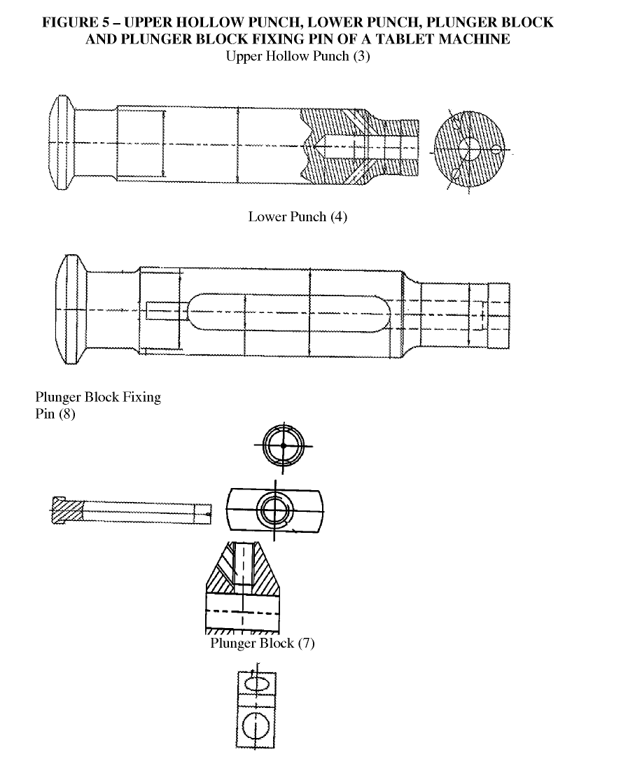

FIG. 5 showing upper hollow punch, lower punch, Plunger block and Plunger block fixing pin of a tablet machine, and

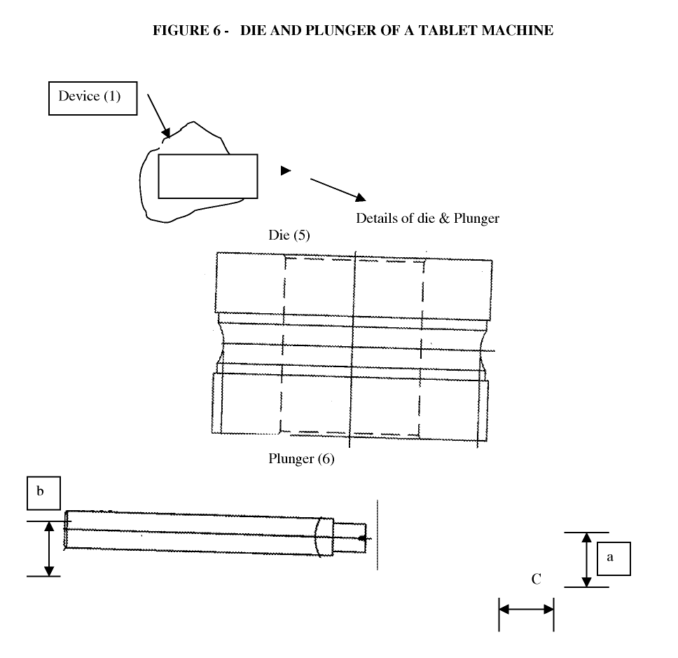

FIG. 6 Die & Plunger of a tablet machine.

The term “modified release” as used herein refers to and includes a prolonged release, extended release, controlled release, controlled delivery, slow release, sustained release and delayed release.

The term “dosage form” as used herein refers to a pharmaceutical preparation in which dose or doses of one or more therapeutically active ingredients are included.

The term “rate controlling polymer” as used herein refers to and includes mean a polymer capable of altering, modulating or modifying rate of release of therapeutically active ingredient from the dosage form. The term “rate controlling polymers” includes pharmaceutically acceptable hydrophilic polymers, hydrophobic polymers, pH independent polymers, pH dependent polymers, soluble polymers, insoluble polymers, lipids or lipidic materials or combinations thereof. The polymer may be a homopolymer or a copolymer.

The inner radial surface of the dosage form according to the invention is partially coated with a polymeric coat comprising on or more of rate controlling polymers. The term partially coated refers to the extent of coating on the inner radial surface such that at least some surface of the inner radial surface remains uncoated.

One way to get the dosage form according to this invention with partially coated inner surface is to vary the ratio of outer radial surface area to the inner radial surface area of the dosage form. In some embodiments, the ratio of outer radial surface area to the inner radial surface area is between 20 to about 1. In some other embodiments, the ratio of outer radial surface area to the inner radial surface area is about 7.

The dosage form according to this invention comprises a core coated with a polymeric coat. The term “core” as used herein refers to a structure that is surrounded by a wall, membrane, or coating. The wall, membrane, or coating can be a functional or non-functional coating. In some embodiments, the core comprises at least one therapeutically active ingredient. In some other embodiments, the core comprises one or more pharmaceutically active excipients. In some embodiments, the core comprises one or more swellable hydrophilic polymers, water soluble and/or insoluble agents.

Typical non-limiting examples of swellable hydrophilic polymers include cellulose derivatives such as hydroxypropylmethyl cellulose (HPMC), hydroxypropyl cellulose, hydroxyethyl cellulose, methyl cellulose, ethyl cellulose, carboxymethyl cellulose, carboxyethyl cellulose, carboxymethylhydroxyethyl cellulose, microcrystalline cellulose, polyethylene oxides, polyvinylpyrrolidone, polyalkylene glycols, gelatine, polyvinyl alcohol, starch and derivatives thereof, acrylic acid polymers, polymethacrylates, polysaccharides such as xanthan gum, tragacanth gum, gum karaya, guar gum, acacia, gellan gum locust bean gum, alkali metal salts of alginic acid or pectic acid, sodium alginate, potassium alginate, ammonium alginate, chitosan, maleic anhydride copolymers, poly(ethyleneimine), polyurethane hydrogels, crosslinked polyacrylic acids, derivatives and the like.

Typical, non-limiting examples of water soluble agents include one or more of lactose anhydrous, lactose monohydrate, mannitol, sodium chloride, sucrose and the like. Typical, nonlimiting examples of water insoluble agents include stearic acid, calcium stearate, magnesium stearate, zinc stearate, sodium stearyl fumarate and the like.

The dosage form according to this invention has a hole extending through the dosage form resulting in an inner radial surface and an outer radial surface. The hole refers to a space, aperture or hollow portion in the dosage form extending through the dosage form and can be of any geometrical shape, including for example, circular, oval, donut, rectangular, triangular, cylindrical, pentagonal, hexagonal, octagonal, trapezoid, combinations thereof and the like. The inner radial surface is the surface surrounding the hole. The outer radial surface refers to the total surface of the dosage form excluding the inner radial surface.

The term “therapeutically active ingredient” as used herein refers to and includes any active pharmaceutical agent or a pharmaceutically acceptable derivative thereof including salts, isomers, mixtures, hydrates, pro-drugs, esters, polymorphs and so on. In some embodiments suitable pharmaceutically acceptable salts include but are not limited to the chloride and other halogen salts, and salts such as are formed by reaction of therapeutically active ingredient with acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pathothenic, phosphoric, p-toluenesulfonic, succinic, sulfuric, and tartaric acids, and the like. The amount of therapeutically active ingredient in the core largely depends on the extent and duration of the therapeutic response desired and can vary depending on various factors, including characteristics of the subject (for example height, weight, sex, age and medical history of the patient). In some embodiment of this invention the dosage form comprises 0.1 to 90% of therapeutically active ingredient.

FIG. 1(a) is a general view of a dosage form designed as donut shaped tablet for oral administration of therapeutically active pharmaceutical ingredient. The regions A and B designate outer surface including upper annular surface and lower annular surface of the dosage form. The inner region extending from C to D designates a hole or hollow portion in the dosage form extending through the dosage form. The outer region extending from E to F designates the outer radial surface of dosage form.

FIG. 1(b) shows the partially coated region extending from C to D of inner surface of donut shaped tablet through which controlled release of therapeutically active pharmaceutical ingredient occurs.

FIG. 2(a) is a general view of a dosage form designed as octagonal shaped tablet for oral administration of therapeutically active pharmaceutical ingredient. The regions A, B and C (all side surfaces) designate the outer surfaces of dosage form. The inner region extending from D to E designates a hole or hollow portion in the dosage form extending through the dosage form.

FIG. 2(b) shows the regions (shown by dotted lines) of inner surface of octagonal shaped tablet through which controlled release of therapeutically active pharmaceutical ingredient occurs.

FIG. 3(a) is a general view of a dosage form designed as triangular shaped tablet for oral administration of therapeutically active pharmaceutical ingredient. The regions A, B, E and F designate the outer surfaces of dosage form. The inner region extending from C to D designates a hole or hollow portion in the dosage form extending through the dosage form.

FIG. 3(b) shows the regions (shown by dotted lines) of inner surface of triangular shaped tablet through which controlled release of therapeutically active pharmaceutical ingredient occurs. The inner radial surface of the dosage form according to this invention is partially coated with a polymeric coat comprising one or more of rate controlling polymers. The term partially coated refers to the extent of coating on the inner radial surface such that at least some surface of the inner radial surface remains uncoated. See, for example, FIG. 1(b) and FIG. 4. FIG. 4 shows the enlarged portion of the inner radial surface showing the partially coated inner radial surface.

In some embodiments, the modified release dosage form according to this invention comprises at least one pharmaceutically acceptable excipient. Typical, non-limiting examples of pharmaceutically acceptable excipients include diluents, binders, disintegrants, super disintegrants, solubilizers, surfactants, enzyme inhibitors, anti-adherents, anticoagulants, antifoaming agents, antioxidants, buffers chelating agents, coagulants, colorants, opaquants, hydrogen bonding agents, flavorants, desensitizers, ion-exchange resins, plasticizers, preservatives, solvents, sweeteners, thickeners, and mixtures.

Typical, non-limiting examples of suitable diluents include microcrystalline cellulose, mannitol, calcium phosphate, calcium sulfate, kaolin, dry starch, powdered sugar and the like.

Typical, non-limiting examples of suitable binders include povidone, starch, stearic acid, gums, hydroxypropylmethyl cellulose and the like.

Typical, non-limiting examples of suitable disintegrants include croscarmellose sodium, crospovidone, sodium starch glycolate and the like.

Typical, non-limiting examples of suitable surfactants include polyoxyethylene glycerol esters of fatty acids, such as Tagats; polyoxylated castor oil, ethylene glycol esters, such as glycol stearate and distearate; propylene glycol esters, such as propylene glycol myristate; glyceryl esters of fatty acids, such as glyceryl stearates and monostearates; sorbitan esters, such as spans and tweens; polyglyceryl esters, such as polyglyceryl 4-oleate; fatty alcohol ethoxylates, such as Brij type emulsifiers; ethoxylated propoxylated block copolymers, such as poloxamers; polyethylene glycol esters of fatty acids, such as Labrafils, Labrafacs, and Labrasols; cremophores; glycerol monocaprylate/caprate, such as Campmul CM 10; Gelucire, Capryol, Captex, Acconon, transcutol, triacetin, TPGS (d-alpha tocopheryl polyethylene glycol succinate) and the like.

Typical, non-limiting examples of suitable lubricants include magnesium stearate, zinc stearate, calcium stearate, stearic acid, sodium stearyl fumarate, hydrogenated vegetable oil, glyceryl behenate and the like.

Typical, non-limiting examples of suitable glidants include colloidal silicon dioxide, talc or cornstarch and the like.

The modified release dosage form according to this invention comprises one or more therapeutically active ingredient. Typical, non-limiting examples of therapeutically active ingredients include Sedatives, Hypnotics, Anti-inflammatory agents, Antibiotics, Antidiabetics, Antihypertensives, Anti-Osteoporosis Agents, Antithrombotic Agents, Antivirals, Antifungals, Anticholinergic Agents, Anxiolytic Agents, Adrenergics, Antipsychotics, Anti-Parkinsonism Agents, Anticonvulsants, CNS Stimulants, Antianginal Agents, Antiarrhythmics, Antihyperlipidemic Drugs, Diuretics, Antiasthmatics, Anticoagulants, Antianemia Agents, Vitamins, Hormones, Antihistaminics, Anticancer Agents, Antiallergics, Antiarthritis Agents, Antialzheimers' Agents, Vasopressin Antagonists, Anticonvulsants, Steroids, Anesthetics, Thrombolytics, Antacids, Proton Pump Inhibitors, Protease Inhibitors, Platelet Aggregation Inhibitors, Mucolytics, Antimalarials, Antiemetics, Laxatives, Expectorants, Enzymes, Contraceptives, Bronchodilators, Antitussives, Antimigraine Agents, Anthelmintics, Anorexiants, and Antiepileptics.

In some embodiments, the therapeutically active ingredient include one or more of Nonlimiting examples of suitable therapeutically active pharmaceutical ingredient may also comprise one or more of Lamotrigine, Amlodipine, Diazepam, Paracetamol, Aspirin, Ciprofloxacin, Dicyclomine, Celecoxib, Alendronate, Diacerein, Acyclovir, Fluconazole, Epinephrine, Divalproex, Methylphenidate, Flecamide, Metoprolol, Fenofibrate, Hydrochlorothiazide, Montelukast, Bicalutamide, Donepezil, Tolvaptan, Saquinavir, Bromhexine, Promethazine, Bisacodyl, Pancreatin, Ethinyl Estradiol, Salbutamol, Diphenhydramine, Sumatriptan, Diclofenac, Metronidazole, Orlistat, Ibuprofen, Indomethacin, Ketorolac, Tramadolol, Oxcarbazepine, Pioglitazone, Rosiglitazone, Miglitol, Vildagliptin, Sitagliptin, Repaglinide, Voglibose, Alprazolam, Chlorpromazine, Cimetidine, Pseudoephedrine, Naproxen, Piroxicam, Atenolol, Benazepril, Captopril, Lisinopril, Fosinopril, Enalapril, Furosemide, Indapamide, Atenolol, Felodipine, Cartenolol, Carvedilol, Cerivastatin, Diltiazem, Fluvastatin, Irbesartan, Candesartan, Methyldopa, Reserpine, Bupropion, Fluoxetine, Paroxetine, Escitalopram, Sertraline, Amitryptiline, Imipramine, Fexofenadine, Clopidogrel, Entacapone, Levodopa, Carbidopa, Levetiracetam, Venlafaxine, Duloxetine, Lisinopril, Losartan, Lovastatin, Niacin, Pravastatin, Ramipril, Simvastatin, Atorvastatin, Valsartan, Telmisartan, Sildenafil, Tadalafil, Vardenafil, Esomeprazole, Famotidine, Omeprazole, Pantoprazole, Rabeprazole, Ranitidine, Simethicone, Artesunate, Amodiaquine, Benazepril, Misoprostol, Metformin, Glipizide, Diltiazem HCl, Verapamil HCl, Labetalol HCl, Theophylline, Diclofenac Sodium, Aceclofenac, Naproxen sodium, Bupropion HCl, Metformin HCl, Duloxetine, Metoprolol tartarate & succinate, Fexofinadine HCL, Pseudoephedrine HCL, Zolpidem tartarate, Tramadol HCl, Oxybutynin chloride, Alfuzosin, including pharmaceutically acceptable salts, hydrates, solvates, esters, prodrugs thereof.

The dosage form according to this invention can be prepared in various forms including, without any limitation, a tablet, minitablets, caplet or pill.

In one of the embodiments of the invention the device (1) comprises tablet machine (2) having an upper hollow punch (3) and a lower punch (4). Lower punch (4) comprises replaceable plunger (6) fitted on to it. The material of construction of the replaceable plunger (6) is such that it could be fitted on to the lower punch (4) easily and also the drug compliant. The plunger (6) size would have to be optimum to ensure compression of the dosage form and also the coating on the aperture portion of the tablet would be partial. It is observed during experimentation that smaller the plunger (6) size, greater is the difficulty of dosage form compression. It is also observed that larger the plunger (6) size, greater is the hole size and the coating would be complete on the surface of the hole portion of the dosage form. Hence an optimum size of the plunger (6) produces a dosage form having a hole which results in partial coating on the surface of the hole portion of the compressed dosage form. FIG. 5 shows upper hollow punch (3), lower punch (4), plunger block (7) and plunger block fixing pin (8).

Distal end of the plunger is that end of the plunger wherein the plunger dimensions are modified or varied as the case may be and corresponds to the shape of the hole formed in the dosage form.

Plunger (6) shown in FIG. 6 may be made of any geometrical shape such as rectangular, triangular, cylindrical, hexagonal, pentagonal, and octagonal, trapezoid and combination of any of them. Plunger dimensions “a” and “c” may be varied so as to create the required size of the hole which ensures partial coating on the inside diameter of the dosage form. Plunger (6) “a” dimension corresponds to lower diameter of the distal end and “b” dimension corresponds to higher diameter of the proximal end of the plunger (6) when the plunger is of cylindrical shape. The length “c” of the lower diameter may extend to such a distance towards the proximal end such that the ratio of “c” to “a” will have correspondence to the ratio of outer radial surface area to the inner radial surface area of the dosage form which is between about 20 to about 1. Within this ratio of the outer radial surface area to inner radial surface area the dosage form will have a partial coating.

When the geometrical shape of the plunger (6) is other than cylindrical, the modification carried out to the distal end of the plunger will have correspondence to the ratio of outer surface area to the inner surface area of the dosage form which is between about 20 to about 1.

The material of construction of the plunger (6) may be made of stainless steel 316 or high chrome, high nickel alloy steel or any other suitable material of construction which is compliant with pharmaceutically active ingredient.

Embodiments of the machine for the manufacture of dosage forms may include one or more of the following features.

In one of the embodiments, device (1) comprises a tablet machine (2) comprising an upper hollow punch (3) and a lower punch (4). A plunger (6) is mounted or fitted on to the lower punch (4). The mounting or fitting may be by means of threaded connection or welding or by means of plunger block (7) and plunger block fixing pin (8). (Plunger (6) passes through the plunger block (7) and the plunger (6) is fixed on to the punch (4) by means of plunger block fixing pin (8).

Plunger (6) comprises higher diameter proximal end and a lower diameter distal end. Depending on the pharmaceutical active ingredient, proximal end or distal end of the plunger (6) may be varied so as to produce a dosage form having a hole of the requisite size. The hole created would be such that the coating on the surface area of hole would be partial. In this embodiment, the plunger (6) is cylindrically shaped as shown in FIG. 6.

In the embodiment of the invention the method of creation of a hole in the dosage form may be as follows. Dosage form comprising therapeutically active pharmaceutical ingredient placed in the die (5) shown in FIG. 6 of tablet machine (2) is compressed by the force of the upper punch (3) while the shape of the plunger (6) corresponds to the creation of a requisite shape of a hole in the dosage form. An angle shape in the plunger block serves for the exit of the spilled pharmaceutical ingredient on the plunger (6) distal end in the embodiment of the present invention.

While the present invention has been described in terms of its specific embodiments, certain modifications and equivalents will be apparent to those skilled in the art and are intended to be included within the scope of the present invention.

1. A device (1) for the manufacture of a dosage form having a hole; said device (1) comprising a tablet machine (2) having a hollow upper punch (3) and a lower punch (4) and a replaceable plunger (6); The replaceable plunger (6) is of any geometrical shape; said device (1) is characterized in that the plunger (6) is fitted on the lower punch (4).

2. The device for the manufacture of the dosage form of claim 1, wherein the plunger (6) serves to create a hole in the dosage form when the compression is carried out.

3. The device for the manufacture of the dosage form of claim 1, wherein the plunger (6) is of any geometrical shape.

4. The device for the manufacture of the dosage form of claim 1, wherein the plunger (6) shape is rectangular, triangular, cylindrical, hexagonal, pentagonal, octagonal and trapezoid and combination of any of them.

5. The device for the manufacture of the dosage form of claim 1, wherein the plunger (6) distal end diameter and its length are “a” and “c”.

6. The device for the manufacture of the dosage form of claim 1, wherein the optimized ratio of “c” to “a” corresponds to the ratio of outer surface area to inner surface area of dosage form is between about 20 to about 1.

7. The device for the manufacture of the dosage form of claim 1, wherein the plunger (6) is fitted on to the lower punch (4) by means of plunger block (7) and plunger bock fixing pin (8).

8. The device for the manufacture of the dosage form of claim 1, wherein the dosage form comprises a therapeutically active ingredient selected from one or more of Sedatives, Hypnotics, Anti-inflammatory agents, Antibiotics, Antidiabetics, Antihypertensives, Anti-Osteoporosis Agents, Antithrombotic Agents, Antivirals, Antifungals, Anticholinergic Agents, Anxiolytic Agents, Adrenergics, Antipsychotics, Anti-Parkinsonism Agents, Anticonvulsants, CNS Stimulants, Antianginal Agents, Antiarrhythmics, Antihyperlipidemic Drugs, Diuretics, Antiasthmatics, Anticoagulants, Antianemia Agents, Vitamins, Hormones, Antihistaminics, Anticancer Agents, Antiallergics, Antiarthritis Agents, Antialzheimers' Agents, Vasopressin Antagonists, Anticonvulsants, Steroids, Anesthetics, Thrombolytics, Antacids, Proton Pump Inhibitors, Protease Inhibitors, Platelet Aggregation Inhibitors, Mucolytics, Antimalarials, Antiemetics, Laxatives, Expectorants, Enzymes, Contraceptives, Bronchodilators, Antitussives, Antimigraine Agents, Anthelmintics, Anorexiants, and Antiepileptics.

9. The device for the manufacture of the dosage form of claim 8, wherein the dosage form comprises a therapeutically active ingredient selected at least from one or more of Lamotrigine, Amlodipine, Diazepam, Paracetamol, Aspirin, Ciprofloxacin, Dicyclomine, Celecoxib, Alendronate, Diacerein, Acyclovir, Fluconazole, Epinephrine, Divalproex, Methylphenidate, Flecamide, Metoprolol, Fenofibrate, Hydrochlorothiazide, Montelukast, Bicalutamide, Donepezil, Tolvaptan, Saquinavir, Bromhexine, Promethazine, Bisacodyl, Pancreatin, Ethinyl Estradiol, Salbutamol, Diphenhydramine, Sumatriptan, Diclofenac, Metronidazole, Orlistat, Ibuprofen, Indomethacin, Ketorolac, Tramadolol, Oxcarbazepine, Pioglitazone, Rosiglitazone, Miglitol, Vildagliptin, Sitagliptin, Repaglinide, Voglibose, Alprazolam, Chlorpromazine, Cimetidine, Pseudoephedrine, Naproxen, Piroxicam, Atenolol, Benazepril, Captopril, Lisinopril, Fosinopril, Enalapril, Furosemide, Indapamide, Atenolol, Felodipine, Cartenolol, Carvedilol, Cerivastatin, Diltiazem, Fluvastatin, Irbesartan, Candesartan, Methyldopa, Reserpine, Bupropion, Fluoxetine, Paroxetine, Escitalopram, Sertraline, Amitryptiline, Imipramine, Fexofenadine, Clopidogrel, Entacapone, Levodopa, Carbidopa, Levetiracetam, Venlafaxine, Duloxetine, Lisinopril, Losartan, Lovastatin, Niacin, Pravastatin, Ramipril, Simvastatin, Atorvastatin, Valsartan, Telmisartan, Sildenafil, Tadalafil, Vardenafil, Esomeprazole, Famotidine, Omeprazole, Pantoprazole, Rabeprazole, Ranitidine, Simethicone, Artesunate, Amodiaquine, Benazepril, Misoprostol, Metformin, Glipizide, Diltiazem HCl, Verapamil HCl, Labetalol HCl, Theophylline, Diclofenac Sodium, Aceclofenac, Naproxen sodium, Bupropion HCl, Metformin HCl, Duloxetine, Metoprolol tartarate & succinate, Fexofinadine HCL, Pseudoephedrine HCL, Zolpidem tartarate, Tramadol HCl, Oxybutynin chloride, Alfuzosin, including pharmaceutically acceptable salts, hydrates, solvates, esters, prodrugs thereof.

10. A method of manufacture of a dosage form having a hole characterized in that the hole created by the device (1) has a partial coating on the surface.
